(19) United States
(12) Patent Application Publication
(10) Pub. No.: US 2011/0009879 A1
(43) Pub. Date: Jan. 13, 2011

(54) APPARATUS FOR STEREOTACTIC NEUROSURGERY

(75) Inventors: Hugo George Derrick, Bristol (GB); Paul David Fielder, Stroud (GB); Steven Streatfield Gill, Bristol (GB)

Correspondence Address: OLIFF & BERRIDGE, PLC, P.O. BOX 320850, ALEXANDRIA, VA, 22320-4850, US

(73) Assignee: RENISHAW (IRELAND) LIMITED, SWORDS (IE)

(21) Appl. No.: 12/733,562

(22) PCT Filed: Oct. 6, 2008
(86) PCT No.: PCT/GB2008/003397

§ 371 Date: Mar. 9, 2010

(30) Foreign Application Priority Data
Oct. 8, 2007 (GB) 0719608.2
Dec. 7, 2007 (GB) 0723880.1

(51) Int. Cl. A61B 19/00

(52) U.S. Cl. 606/130

(57) ABSTRACT

A skull mount (50;150;170;200;300) is described that is attachable to a hole (60) formed in the skull. The skull mount (50;150;170;200;300) comprises an alignment guide (62;152;172;216;306) defining an alignment axis (22;210;312) along which neurosurgical instruments can be passed. The skull mount, when attached to a hole in a skull, is arranged such that it does not substantially protrude from the outermost surface of the skull and does not extend into the brain parenchyma. Also described is a neurosurgical alignment instrument (30,206) for aligning such a skull mount (50;150;170;200;300) that comprises an elongate shaft (32) and an element (34,36) protruding from the distal end of the elongate shaft (32) for engaging and aligning the alignment guide (62;152;172;216;306) of an associated skull mount (50;150;170;200;300). When the alignment instrument is engaged with a skull mount attached to a hole formed in the skull, the protruding element passes through the alignment guide of the skull mount and into the cortex of the subject's brain.

The present invention relates to apparatus for use in neurosurgery and to methods of neurosurgery. In particular, the present invention relates to apparatus and methods for use in stereotactically targeted treatment of abnormalities of brain function, and for accurately guiding instruments directly into the brain parenchyma.

There are many situations where there is a requirement to deliver therapeutic agents to specific targets within the brain parenchyma via implanted catheters. Furthermore, many of these therapeutic agents will cause unwanted side effects if delivered to healthy parts of the brain. Examples of treating abnormalities of brain function include the acute infusion of Gamma-amino-buturic-acid agonists into an epileptic focus or pathway to block transmission, and the chronic delivery of opiates or other analgesics to the peri-aqueductal grey matter or to thalamic targets for the treatment of intractable pain. Also, cytotoxic agents can be delivered directly into a brain tumour. Intraparenchymal infusion can also be used to deliver therapeutic agents to brain targets that can not be delivered systemically because they will not cross the blood-brain barrier. For example, the treatment of patients with Parkinson's disease, Alzheimer's disease, head injury, stroke and multiple sclerosis may be carried out by the infusion of neurotrophic factors to protect and repair failing or damaged nerve cells. Neurotrophins may also be infused to support neural grafts transplanted into damaged or malfunctioning areas of the brain in order to restore function.

It is also known to insert instruments other than catheters, such as electrodes, directly in the brain parenchyma. For example, stimulating and lesioning electrodes are used in a variety of surgical procedures, including deep brain stimulation (DBS) electrodes. A surgeon wishing to stimulate or lesion a particular area of nervous tissue can target the end of an electrode to the target site so that a desired electrical current can be delivered.

The above described methods rely on targeting the required site as accurately as possible. Sub-optimal placement of the instrument being inserted may lead to significant morbidity or treatment failure. For example, brain targets for treating functional disorders are usually deeply situated and have small volumes. A desired target for treating Parkinson's disease is situated in the sub-thalamic nucleus and is 3-4 mm in diameter, or an ovoid of 3-4 mm in diameter and 5-6 mm in length. Other targets such as the globus palladus or targets in the thalamus are usually no more than 1-2 mm larger. For such a small target sub-optimal placement of as little as 1 mm will not only reduce the effectiveness of the treatment, but may also induce unwanted side affects such as weakness, altered sensation, worsened speech and double vision. It is also desirable to minimise trauma in certain regions of the brain; for example, the mesencephalon (which includes the subthalamic nucleus, the substantia nigra and the pedunculor-pontine nucleus) is a critical region of the brain where is it is important to minimise trauma from the passage of an electrode or catheter.

A variety of stereotactic devices and methods have thus been developed previously in an attempt to allow instruments to be accurately guided towards a target identified by a surgeon (e.g. using x-rays or magnetic resonance imaging) with the minimum of trauma to other regions of the brain. Examples of prior systems are given in EP1509153, U.S. Pat. No. 6,609,020 and U.S. Pat. No. 6,328,748.

U.S. Pat. No. 6,609,020 describes an elongate guide tube having a threaded head for attachment to a burr hole formed in a skull. EP 1509153 describes a stereoguide that is fixable to a stereotactic frame that includes a stereotactic base ring secured to a subject's skull by a plurality of screws. The stereoguide of EP1509153 comprises two guide members that provide an axis of insertion through which instruments may be passed. Two clamps are also provided on the stereoguide to allow the instruments to be clamped as required. Such an arrangement allows the insertion of catheters, electrodes or guide tubes of the type described in U.S. Pat. No. 6,609,020 to identified targets in the brain. Although the arrangement of EP1509153 typically provides reliable instrument positioning, moving the various clamps into and out of position can sometimes be a somewhat involved and time consuming process for a surgeon.

It is also known, as an alternative to attaching a stereotactic frame to a subject, to attach a lockable ball joint assembly to the outer surface of the skull of a patient. For example, U.S. Pat. No. 6,328,748 describes a guide that comprises a holder formed from a lower ring and an upper ring that, when assembled together, capture a ball held on a stalk that has a channel through which medical instruments can be passed. The lower ring also comprises an external threaded surface that can be screwed into a burr hole formed in a patients skull. In use, the lower ring is attached to the skull and the ball inserted therein. The upper ring is then screwed onto the lower ring to capture the ball. An alignment tool is then inserted through the stalk and into the ball and aligned along a required axis of insertion with the aid of a stereotactic pointer. Once the required alignment has been set, the upper ring is screwed further into engagement with the lower ring thereby locking the ball in position and fixing the orientation of the channel provided through the ball. Instruments may then be inserted through the ball along the required axis of insertion to obtain biopsy material or the like. Such instruments are then withdrawn from the subject and the instrument guide is unscrewed from the burr hole and removed from the subject. Although devices of this type are simpler for a surgeon to use than a stereotactic frame based system, they can not typically achieve the same levels of targeting accuracy that are possible with stereotactic frame based techniques.

According to a first aspect of the present invention, a skull mount is provided that is attachable to a hole formed in the skull of a subject, the skull mount comprising an alignment guide defining an alignment axis along which neurosurgical instruments can be passed, characterised in that the skull mount, when attached to a hole in a skull, does not substantially protrude from the outermost surface of the skull and does not extend into the brain parenchyma.

The present invention thus provides a skull mount that can be located within or substantially within an aperture or hole formed in the skull of a subject. The skull mount comprises an alignment guide or guide member, such as a channel or passageway, that defines an alignment axis along which neurosurgical instrument, such as tubes or wires, can be passed. As outlined in more detail below, the alignment axis of the alignment guide of the skull mount can be adjusted to coincide with a required (e.g. predetermined) axis of neurosurgical instrument insertion. The skull mount does not substantially protrude from the outermost surface of the skull; e.g. the proximal end of the skull mount may be located mostly or substantially within or below the skull bone to which it is attached such that it does not protrude by a significant amount from the outer surface of the skull. Furthermore, the skull mount does not extend into the brain parenchyma. In other words, the distal end of the skull mount is arranged to protrude only a short distance, if at all, into the skull cavity such that there is no significant portion of the skull mount located within the brain parenchyma.

Advantageously, the skull mount is arranged such that, when inserted in a hole formed in the skull of a subject, it is substantially flush to the outermost surface of the skull. The skull mount may not protrude at all from the skull or may even be located completely below the skull surface (e.g. it may be sub-flush to the skull). In a preferred embodiment, the skull mount protrudes from the outer skull surface by no more than 1 cm, more preferably by no more than 5 mm and more preferably by no more than 3 mm.

The other dimensions of a skull mount of the present invention will depend on the thickness of the skull bone and may vary from subject to subject and for different species. To avoid contact with the brain parenchyma, it is preferred that the skull mount extends no more than approximately 5-10 mm into a human skull cavity. The skull bones of an average human range in thickness from around 6 mm to 10 mm; although it is not uncommon for there to be variations of several millimetres outside of this range. It is thus preferred that the skull mount extends into the skull from the outer surface of the skull by no more than 20 mm, more preferably by no more than 15 mm, more preferably by no more than 10 mm, more preferably by no more than 8 mm and more preferably by no more than 5 mm. It can thus be seen that the preferred length of the skull mount along the axis of insertion is no more than 3 cm, more preferably no more than 2 cm and more preferably no more than 1 cm.

A skull mount of the present invention does not protrude a substantial amount from the skull and can therefore, if required, remain implanted in a subject after a surgical procedure has been performed. For example, the present invention permits a skull mount to be provided that is suitable for long term, subcutaneous, implantation within a subject. This should be contrasted to devices of the type described in U.S. Pat. No. 6,328,748 that are designed for short term attachment to a subject (e.g. to collect biopsy samples) and are detached from the subject after completion of the required surgical procedure and prior to removal of the subject from the sterile environment of the operating theatre. Skull mounts of the type described in U.S. Pat. No. 6,328,748 are predominantly located outside of the skull and would be unsuitable for long term implantation as they could not be buried subcutaneously and would therefore pose a substantial risk of channeling infection into the brain if left attached after surgery. It should be noted that, as described below, a skull mount of the present invention is particularly suitable for use with a stereoguide and, in a preferred embodiment, the alignment axis of the alignment guide of the skull mount may be aligned with an axis of instrument insertion defined by the stereoguide. Instruments may then be inserted into the brain parenchyma with guiding providing by both the stereoguide and the skull mount. A skull mount of the present invention can thus be seen to also improve the targeting accuracy of stereoguide based neurosurgical apparatus.

As noted above, the skull mount is advantageously suitable for long term, subcutaneous, implantation within a subject. Long term implantation may mean the skull mount remaining with the body for weeks, months or even years at a time; i.e. long after the initial surgical intervention. In such a case, the skull mount is conveniently formed from materials that are suitable for long term implantation within the body. For example, the skull mount may be formed from a plastic material such as Barex (Trademark), PEEK (Polyaryletheretherketone) or a thermoplastic polyurethane elastomer (TPU) such as carbothane (Trademark). The skull mount is conveniently fabricated from a material that is opaque to x-rays or is detectable using MRI so that it can be readily identified after implantation. Conveniently, the skull mount comprises only non-magnetic material so that a patient with the mount implanted therein can be safely subjected to an MRI scan. As outlined in more detail below, the implanted skull mount may be provided as part of a long term implanted drug delivery or deep brain stimulation system.

Preferably, the alignment guide of the skull mount comprises a member having a channel formed therethrough defining the alignment axis. The orientation of the skull mount within a hole in the skull can then be adjusted during attachment of the skull mount to the skull to align the alignment axis with the required axis of neurosurgical instrument insertion. In other words, the skull mount may have a channel having a fixed location relative to the rest of the skull mount. The orientation of the skull mount within a hole formed in a skull may then be adjusted to provide the required alignment of the alignment axis. The aligned skull mount may then be fixed in the skull hole with an adhesive, such as Cyanoacrylate, Polymethyl methacrylate (PMMA) or a UV curable adhesive. A layer of such adhesive may also, or alternatively, provide the alignment guide itself; e.g. by curing the adhesive so as to form a channel co-axial with the alignment axis. The skull mount may also be fixed in place by a press-fit attachment.

Alternatively, the alignment guide of the skull mount may conveniently comprise a member defining the alignment guide and a socket attachable to a hole formed in a subject's skull. The member defining the alignment guide may be moveable relative to, and optionally retained by, the socket. In such an example, the socket may be provided as an integral part of the skull mount and may be locatable substantially within a hole formed in a subject's skull. The socket may have a lip or rim that is larger than the underlying socket portion in which the ball is located. The rim may then sit on, and be attached (e.g. screwed) to, the outer surface of the skull whilst the socket portion is substantially located within or below the hole formed in the skull. In a preferred embodiment, the moveable member providing the alignment guide may comprise a ball or similar that has a channel formed therethrough to define the alignment axis. The ball may be retained within the socket.

Preferably, the moveable member (e.g. the ball) can be immobilised relative to the socket thereby allowing the alignment axis to be fixed or locked in place. For example, an adhesive may be used to lock the ball in position relative to the socket after alignment of the skull mount. Alternatively, a releasable locking mechanism (such as a locking screw) may be provided to immobilise the ball relative to the socket when required. An arrangement of this type allows the skull mount to be implanted within the hole formed in the skull using, for example, an adhesive, a press-fit attachment or a screw-fit attachment. Once the socket is attached to the skull, an alignment process may be used to align the alignment axis defined by the moveable member (e.g. the ball) of the socket. The moveable member may then be locked in place within the socket after alignment. Such a post-attachment alignment technique would simply not be possible using stereotactically inserted guide tubes of the type described in U.S. Pat. No. 6,609,020.

An alternative ball and socket arrangement may be provided in which the socket is, at least partially, formed by a suitably shaped hole formed in the skull of a subject. For example, a socket may be provided that includes a recess formed in the skull that has an upper part comprising a chamber in which the ball is located and a lower part that comprises a recess having a smaller cross section against which the ball is seated. A capping portion may also be provided that can be screwed in place on the surface of the skull to retain the ball within the chamber.

If the alignment guide is provided in the form of a channel as described above, the skull mount may also comprise a fluidic seal to prevent any fluid passing through the channel when no neurosurgical instruments are present in the channel and/or to provide a seal against an inserted instrument. For example, the channel may include a septum seal or similar to seal the channel when access to the brain is not required. A separate sealing cap may also be provided that is attachable to the skull mount (e.g. when no neurosurgical instruments are inserted through the skull mount) to provide a fluidic sealing function.

Advantageously, the skull mount comprises a recess or other suitable feature that allows releasable attachment of the skull mount to a neurosurgical alignment instrument. A neurosurgical alignment instrument may thus hold the skull mount during the procedure of attaching the skull mount to a hole formed in a subject's skull. The surfaces of the skull mount defining the recess preferably carry a screw thread for releasable attachment to a complimentary protrusion provided on that associated neurosurgical alignment instrument. The recess may be co-axial with the alignment guide of the skull mount. In this manner, the skull mount may be screwed onto a neurosurgical alignment instrument, such as an instrument according to the second aspect of the invention as described below.

Conveniently, after stereotactic implantation, a surface of the skull mount provides a fixed reference position or datum marker. For example, the position of an outermost surface of the skull mount may be measured along the axis of insertion relative to a reference point on the stereotactic frame. The position of a brain target along the axis of insertion may also be known relative to the reference point on the stereotactic frame. It thus follows that the distance from the reference surface of the skull mount to the brain target can be readily determined and the depth of insertion of neurosurgical instruments can subsequently be measured relative to the skull mount reference surface.

It should be remembered that it is only the skull mount that does not substantially protrude from the surface of the skull or enter the brain parenchyma. The whole purpose of the skull mount, when implanted, is to guide other neurosurgical instruments (e.g. catheters, electrodes, guide tubes) to one or more desired targets within the brain. Furthermore, the process of implanting the skull mount may result in some penetration of the brain parenchyma and/or may temporarily require a structure to protrude outwardly from the skull. For example, as described below, a separate neurosurgical alignment instrument may be used to attach the skull mount using a stereotactic frame; this alignment instrument may also penetrate the dura and possibly forge a passageway through the cortex. It would also be possible to provide a detachable implantation member(s) that is attached to the skull mount during implantation but subsequently detached therefrom. For example, the skull mount may be attached to and/or formed integrally with an implantation member (e.g. an elongate tube that is co-axial with the alignment axis) that is used during the implantation process. The implantation member may be inserted into the brain, or protrude outwardly from the skull, during the skull mount implantation process. The implantation member may then be detached from the skull mount (e.g. it may be snapped or cut from the skull mount) after implantation and withdrawn from the subject.

According to a second aspect of the present invention, a neurosurgical alignment instrument is provided for aligning a skull mount, the skull mount being attachable to a hole formed in the skull of a subject and including an alignment guide defining an alignment axis along which neurosurgical instruments can be passed, the instrument comprising; an elongate shaft and an element protruding from the distal end of the elongate shaft for engaging and aligning the alignment guide of an associated skull mount; characterised in that, when the instrument is engaged with a skull mount attached to a hole formed in the skull of a subject, the protruding element passes through the alignment guide of the skull mount and penetrates the cortex of the subject's brain.

A neurosurgical alignment instrument is thus provided for aligning the alignment axis of a skull mount, such as a skull mount according to the first aspect of the present invention. The alignment instrument comprises an elongate shaft having a protruding element at its distal end that can engage the alignment guide of an associated skull mount, such as a skull mount according to the first aspect of the invention. In addition to providing an alignment function, the distal end of the protruding element of the instrument is arranged to pass completely through the alignment guide of the skull mount. When the skull mount is attached or is being attached to a hole formed in the skull, the distal end of the protruding element passes through the alignment guide and into the brain cortex, optionally penetrating the dura. Unlike alignment devices of the type described in U.S. Pat. No. 6,328,748 (e.g. see pointer 19 shown in FIG. 2 of U.S. Pat. No. 6,328,748), the alignment instrument of the present invention performs a dual role of aligning the alignment axis of the skull mount and also entering the brain cavity to form an pathway through the brain tissue (e.g. by forcing a path through the dura and/or cortex).

Advantageously, the elongate shaft of the alignment instrument is appropriately dimensioned such that it can be guided along a required axis of insertion by an associated stereoguide. The elongate shaft may, for example, be of substantially circular cross-section and have a constant radius along its length. The elongate shaft may be formed from a resilient material, such as stainless steel, that exhibits a minimal amount of distortion during use. The associated stereoguide may hold the alignment instrument such that the central longitudinal axis of the elongate shaft of the instrument lies substantially along the axis of insertion that is defined by the stereoguide as it is moved towards the skull of the subject. In a preferred embodiment, the stereoguide comprises two or more alignment guides for guiding the elongate shaft of the alignment instrument.

Conveniently, the protruding element is substantially co-axial with the longitudinal axis of the elongate shaft. In this manner, the protruding element may be passed through the alignment guide of the skull mount (thereby aligning the alignment axis of the mount with the axis of insertion defined by the stereoguide) and forced into contact with the brain of the subject from a direction that corresponds to the axis of insertion defined by the stereoguide. The protruding element advantageously comprises a length of wire; for example, the protruding element may be formed from a length of wire having an outer diameter of 0.5 mm to 1.5 mm (e.g. 1 mm). The distal end of the protruding element may comprise a sharp tip for piercing the dura. Preferably, the protruding element is arranged to penetrate between 10 mm to 12 mm into the brain thereby not only piercing the dura but also forming a passageway through the cortex. As explained in more detail below, the brain tissue underlying the cortex is generally significantly softer than the cortex and dura. The alignment instrument of the present invention can thus be seen to forge a passage through the toughest, outermost, layers of the brain thereby easing any subsequent introduction of a guide wire and/or guide tube into the softer tissue underlying the cortex.

Advantageously, an attachment member is provided at the distal end of the elongate shaft, the attachment member being releasably engageable with an associated skull mount. The attachment member may comprise, for example, a threaded protrusion or stump that is co-axial with the protruding member and elongate shaft. This allows a skull mount to be attached (e.g. screwed) to the end of the alignment instrument and then passed along the axis of insertion and into engagement with the hole formed in the skull. The skull mount may then be affixed to the skull hole using an adhesive; the alignment instrument ensuring that the alignment axis of the skull mount is kept in alignment with the insertion axis defined by the stereoguide whilst the adhesive cures. It should be noted that the attachment member is by no means essential. For example, the alignment instrument may be used to align a skull mount (e.g. a ball and socket type skull mount as described above) that has already been attached to the skull.

Preferably, a plurality of scale markings are provided on the elongate shaft. Providing such markings allows the distance between the distal end of the elongate shaft and a point on the stereoguide to be measured. This distance information can then be used to calculate the distance from the skull mount to the desired brain target along the axis of insertion thereby enabling the length of any subsequently inserted neurosurgical instruments (e.g. guide wires, guide tubes, catheters etc) to be precisely calculated.

According to a third aspect of the invention, an applicator instrument for inserting a guide wire directly into the brain parenchyma of a subject is provided, characterised in that the instrument comprises an elongate shaft having a hollow channel for retaining a guide wire, the hollow channel being substantially co-axial with the longitudinal axis of the elongate shaft, wherein, in use, a guide wire is retained by the hollow channel and arranged to protrude therefrom such that, when the instrument is moved along an axis of insertion towards a subject, the distal end of the guide wire is also moved along the required axis of insertion.

The present invention thus provides an applicator instrument for inserting a guide wire directly into the brain parenchyma of a subject. The applicator instrument is particularly suitable for inserting a guide wire through a skull mount according to the first aspect of the invention that has had its alignment axis aligned with a required axis of insertion using a neurosurgical alignment instrument according to the second aspect of the invention. The applicator instrument comprises an elongate shaft having a centrally located hollow channel running along its length. Advantageously, the elongate shaft is rigid and is dimensioned such that it can be guided along a required axis of insertion by an associated stereoguide. The hollow channel is arranged to receive and retain a guide wire and, in use, to have a length of guide wire protruding therefrom. Conveniently, a clamp is provided to prevent longitudinal movement of a guide wire when retained by the instrument. The applicator instrument is arranged such that, in use, movement of the instrument by a stereoguide along the axis of insertion drives the protruding wire along the required axis of insertion and in to the brain parenchyma.

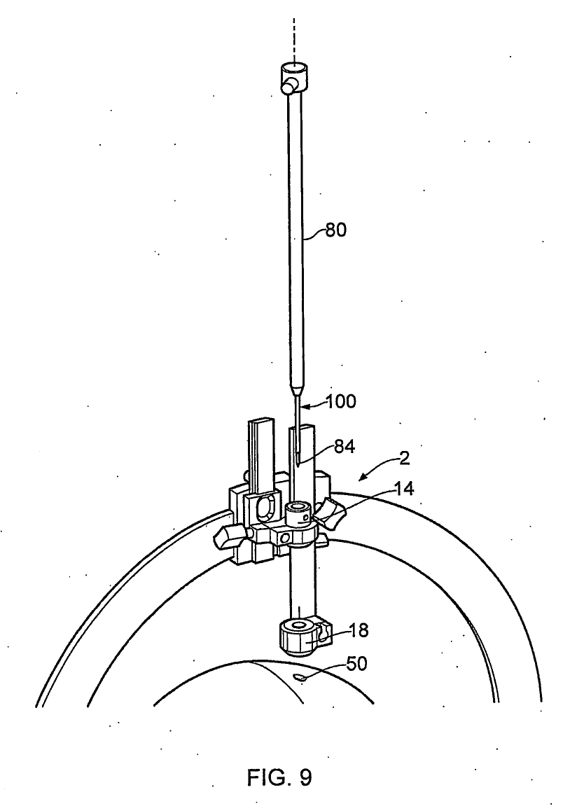

Preferably, the distal end of the elongate shaft comprises a feature or features for engaging a neurosurgical instrument. For example, the feature may comprise a recess or protrusion for engaging (e.g. by a frictional fit) a corresponding feature of the neurosurgical instrument, Conveniently, the feature may comprise a recess that is shaped for releasably engaging the hub of a guide tube. For example, the elongate shaft may be arranged to engage the hub of the guide tube described in WO03/07785 and shown in FIGS. 8 and 9 thereof.

Advantageously, the hollow core of the applicator instrument has a substantially circular cross-section. A guide wire having a substantially circular cross-section may also be provided that is retained within the hollow core. The outer diameter of the guide wire and the internal diameter of the hollow channel are preferably selected such that the guide wire can be slideably retained within the channel without any substantial relative radial movement between the guide wire and the elongate shaft. In other words, the wire preferably fits snugly within the hollow channel. A suitable lubricant may also be provided to facilitate insertion of the wire into the hollow channel, if required.

According to a fourth aspect of the invention, neurosurgical apparatus comprises; a stereoguide for guiding neurosurgical instruments along a defined axis of insertion; a skull mount comprising an alignment guide having an alignment axis; and a skull mount alignment instrument for aligning the alignment axis of the skull mount; wherein, in use, the skull mount alignment instrument is carried by the stereoguide and aligns the alignment axis of the skull mount with the axis of insertion defined by the stereoguide.

The present invention thus provides neurosurgical apparatus comprising a skull mount that can be attached to a hole formed in the skull of a subject. The apparatus also includes a skull mount alignment instrument for aligning the alignment axis of the skull mount relative to the skull to which it is attached and a stereoguide for carrying the neurosurgical instrument. In use, the skull mount alignment instrument is carried by the stereoguide and allows the alignment axis of the skull mount to be aligned with the axis of insertion that is defined by the stereoguide. In this manner, an additional or tertiary guiding element is provided near the surface of the brain by the skull mount thereby enabling neurosurgical instruments (e.g. guide wires, guide tube etc) to be moved along the required axis of insertion with guidance from both the stereoguide and from the skull mount. In this manner, neurosurgical instruments can be driven along the desired axis of insertion into the brain parenchyma with a higher level of accuracy than would be possible using a stereoguide or skull mount based system alone.

After insertion and alignment of the skull mount, a guide wire may be inserted into the brain parenchyma through the skull mount with guidance from the stereoguide. The apparatus thus conveniently comprises an applicator instrument for retaining a guide wire. In use, the applicator instrument may be carried by the stereoguide to allow a guide wire to be passed through the alignment guide of an implanted skull mount and into the brain parenchyma of a subject, the stereoguide and the alignment guide of the skull mount acting so as to guide the guide wire along the defined axis of insertion. In a preferred embodiment, the applicator instrument may conveniently comprise an instrument according to the third aspect of the invention.

Advantageously, the applicator instrument is arranged to insert a guide wire surrounded by a guide tube into the brain parenchyma.

Any skull mount having an alignment guide that can be adjusted so that its alignment axis corresponds to the required axis of insertion may be used. Preferably, the apparatus comprises a skull mount according to the first aspect of the present invention that does not substantially protrude from the skull surface. Similarly, any type of appropriate skull mount alignment instrument may be used in combination with the stereoguide, although the skull mount alignment instrument is preferably an instrument according to the second aspect of the invention. The skull mount alignment instrument may also be arranged to carry and insert the skull mount into the hole formed in the skull.

Advantageously, the stereoguide comprises two or more alignment guides for guiding neurosurgical instruments, such as the skull mount alignment instrument and/or the applicator instrument, along a defined axis of insertion. If appropriate, the alignment guides of the stereoguide may be fitted with different inserts for guiding instruments of different dimensions. The stereoguide may thus comprise at least a first alignment guide and a second alignment guide for guiding a neurosurgical instrument, the first and second alignment guides providing an axis of insertion for neurosurgical instruments. Advantageously, stereotactic frame is provided that includes the stereoguide and a base ring, the base ring being directly attachable to the skull of a subject. For example, the stereotactic frame of the type sold by Elekta may be used. A localiser box having a plurality of fiducial markers may also be separately mountable to the base ring thereby allowing a required axis of insertion to be established using an imaging technique (e.g. MRI) and then related to the stereoguide position.

The apparatus may further comprise at least one of a guide wire, a catheter, a guide tube, an electrode and a biopsy needle. The catheter, guide tube and/or electrode may be suitable for long term implantation within a subject and may thus form part of an implanted drug delivery or deep brain stimulation system.

According to a fifth aspect of the invention, a method for aligning a skull mount relative to a hole formed in a subject's skull is provided, the skull mount comprising an alignment guide defining an alignment axis along which neurosurgical instruments can be passed, the method comprising the step of (i) using a stereoguide to align said alignment axis with a predetermined axis of insertion. Preferably, the skull mount is a skull mount according to the first aspect of the invention.

The method of the present invention thus provides a procedure for accurately aligning the alignment axis of a skull guide using a stereoguide. Unlike previous skull mounts of the type described in U.S. Pat. No. 6,328,748, the use of a stereoguide to provide skull mount alignment enables higher accuracy alignment to be achieved.

Conveniently, step (i) comprises the step of using a stereoguide that forms part of a stereotactic frame that is mounted to the subject's skull. The stereotactic frame may also comprise a stereotactic base ring that can be securely affixed to the subject's skull using screws or the like. As explained above, the stereoguide may be releasably attached to the stereotactic base ring. In this manner, the stereoguide is separately mounted to the skull of the subject and is not supported or aligned in any way by the skull mount.

Advantageously, step (i) is preceded by a step of configuring the stereoguide so as to guide neurosurgical instruments along the predetermined axis of insertion. For example, the stereoguide may have at least two alignment guides that define an axis of insertion along which neurosurgical instruments may be passed. The step of configuring the stereoguide may then comprise setting the at least two alignment guides so that the stereoguide can guide neurosurgical instruments along the required axis of insertion.

Conveniently, step (i) is preceded by the step of determining the axis of insertion along which neurosurgical instruments are to be guided to a desired target in the brain parenchyma. The axis of insertion may be found, for example by a surgeon, from diagnostic images acquired of the subject's brain. The step may thus be performed of imaging the subject's head, for example using MRI or an X-ray based device, and determining the desired brain target and axis of instrument insertion from the acquired images. The imaging step may also include the step of attaching a so-called localiser box to a stereotactic base ring that is in turn attached to the subject's head as described above. The localiser box is advantageously repeatably attachable to the base ring and contains a plurality of fiducial markers thereby enabling the co-ordinates of targets identified from the image to be measured relative to the base ring. The stereoguide may also be affixed to the base ring in a known, repeatable, location after removal of the localiser box and may thus be positioned to provide the axis of instrument insertion as determined by a surgeon from the acquired images.

Advantageously, step (i) comprises using the stereoguide to guide a neurosurgical alignment instrument along the predetermined axis of insertion, the neurosurgical alignment instrument comprising an elongate shaft and an element protruding from the distal end thereof. The neurosurgical alignment instrument used in this step may be an instrument according to the second aspect of the invention. Step (i) may then further comprise bringing the protruding element of the neurosurgical alignment instrument into engagement with the alignment guide of the skull mount, thereby aligning the alignment axis of the skull mount with the predetermined axis of insertion. Furthermore, the distal end of the protruding element of the neurosurgical alignment instrument is preferably arranged to pass through the alignment guide of the skull mount, wherein step (i) may then comprise the step of fording the distal end of the protruding element in to the subject's brain cortex, optionally piercing the dura in the process. The method of the present invention may thus employ the neurosurgical alignment instrument to not only align the alignment guide but to also penetrate or pierce the dura of the subject and/or provide deeper penetration, e.g. into the brain cortex, if required.

The skull mount may be attached to the hole formed in the subject's skull and then aligned. Advantageously, the skull mount is both aligned and attached to the hole in a single action. Step (i) may thus comprise using the neurosurgical alignment instrument to carry a skull mount along the axis of insertion and into engagement with the hole formed in the subjects skull. The dura may be pierced before step (i) or as the skull mount is brought into engagement with the hole formed in the skull.

After the skull mount has been inserted and aligned, the orientation of the alignment axis of the skull mount may be locked in position. A step (ii) of fixing the orientation of the alignment axis of the alignment guide of the skull mount may thus follow the alignment step (i).

Once the skull mount has been implanted and aligned, the method conveniently comprises the step (iii) of using the stereoguide to pass a guide wire, optionally inserted into a guide tube, through the alignment guide of the skull mount and along the predetermined axis of insertion into the brain parenchyma; Step (iii) may be conveniently performed using an applicator instrument according to the third aspect of the invention. Passing such a wire through the aligned alignment guide of the skull mount improves the accuracy with which the wire follows the axis of insertion.

As noted above, step (iii) may include inserting a guide wire inserted through a guide tube in the brain parenchyma. In such a case, a step (iv) may be performed of withdrawing the guide wire from the subject whilst leaving the guide tube in situ. The guide wire can thus be seen to provide rigidity to ensure the guide tube follows the required axis of insertion. Once the guide tube is properly aligned, the guide wire may be withdrawn back through the guide tube. Conveniently, the guide tube may have a hub at its proximal end connectable to the skull mount. The step of inserting the guide wire and the guide tube may thus comprise attaching (e.g. screwing, clipping or snap/press fitting) the guide tube to the skull mount. In this manner, the guide wire can be withdrawn without causing any displacement of the guide tube. Once the guide tube is implanted, neurosurgical instruments may be passed along the guide tube to the identified brain target. For example, a step (v) may be performed of inserting at least one of an intraparenchymal catheter and an intraparenchymal electrode into the brain parenchyma through the guide tube.

The hole formed in the subject's skull for receiving the skull mount may be provided by any technique. Advantageously, step (i) is preceded by the step of using a drill bit to drill a hole in the skull of the subject, wherein the stereoguide is used to pass the drill bit along the predetermined axis of insertion into contact with the subject's skull. In this manner, the hole may also be aligned with the axis of insertion.

It should be noted that although the description contained herein is predominantly directed to method and apparatus for inserting intracranial catheters for delivering therapeutic agents, the invention can also be used in other applications. For example, catheters may be implanted to drain fluid from the brain or electrodes may be inserted for deep brain stimulation. A person skilled in the art would also recognise the various other uses of the apparatus and methods described herein.

The invention will now be described, by way of example only, with reference to the accompanying drawings in which;

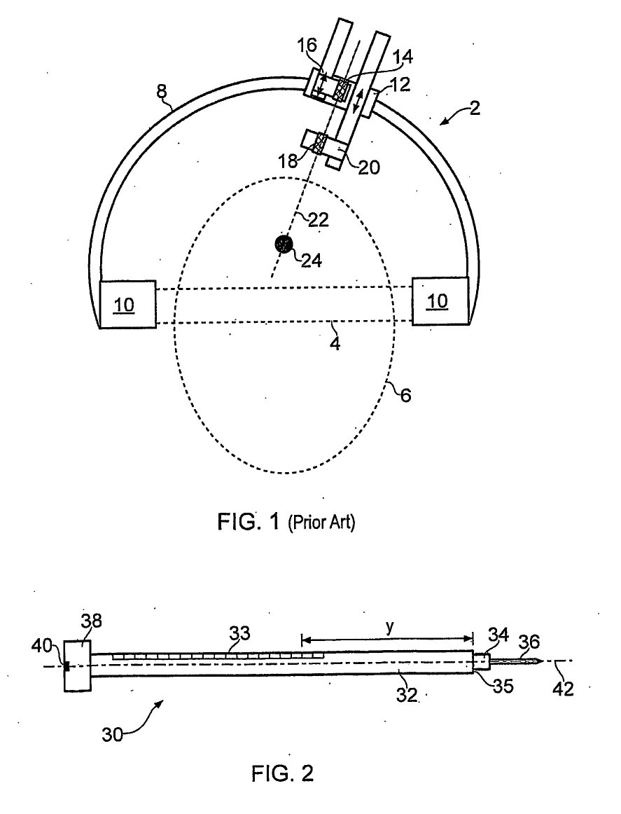

FIG. 1 shows a known stereoguide frame,

FIG. 2 illustrates a skull mount insertion and alignment device,

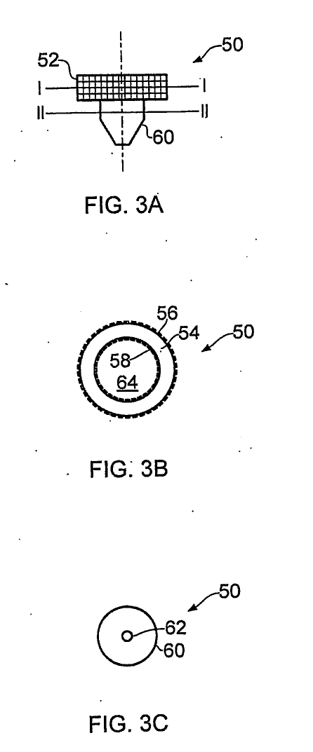

FIGS. 3a-3c show a skull mount,

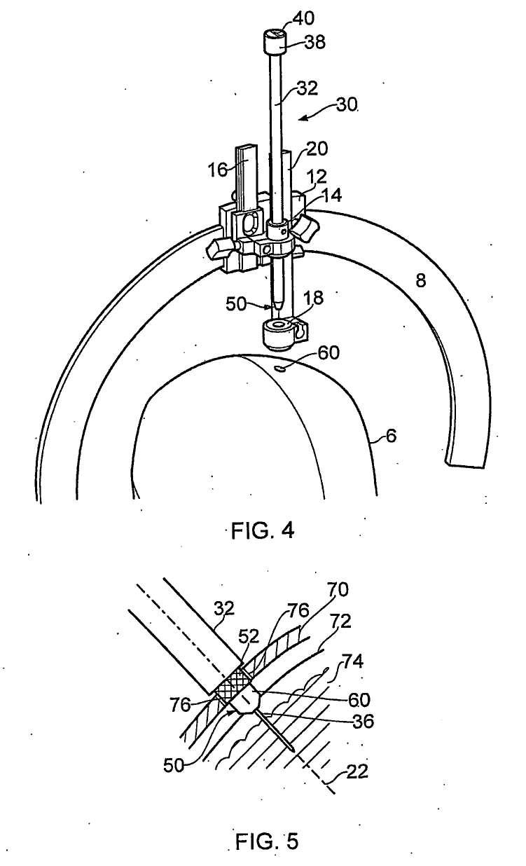

FIG. 4 illustrates the skull mount insertion and alignment device of FIG. 2 carrying a skull mount of FIG. 3 and attached to a stereoguide frame of FIG. 1,

FIG. 5 shows the skull mount insertion and alignment device when fully engaged with the skull,

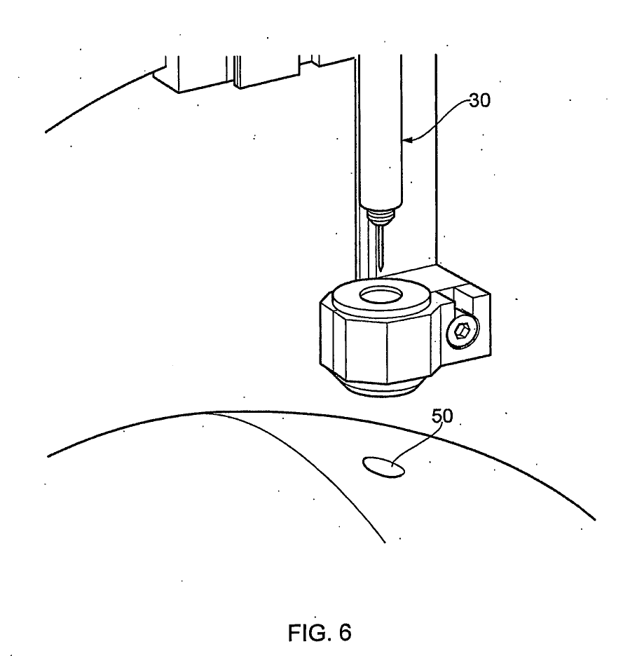

FIG. 6 shows a skull mount after retraction of the skull mount insertion and alignment device,

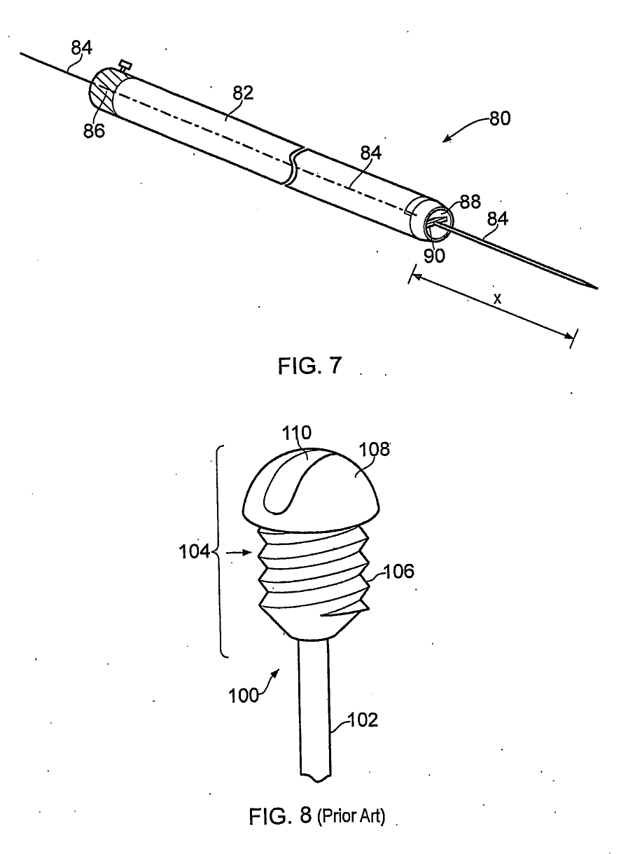

FIG. 7 illustrates a guide tube applicator retaining a length of guide wire,

FIG. 8 illustrate a plastic guide tube having a slotted hub,

FIG. 9 illustrates the guide tube applicator, guide wire and guide tube prior to insertion into the skull mount,

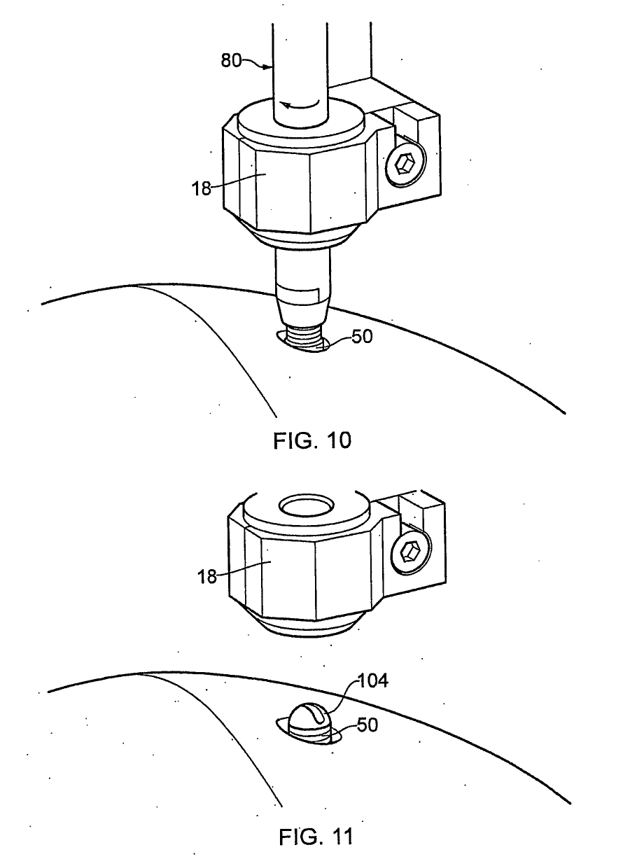

FIG. 10 illustrates engagement of the guide tube hub and skull mount device,

FIG. 11 illustrates the guide tube when attached to the skull mount,

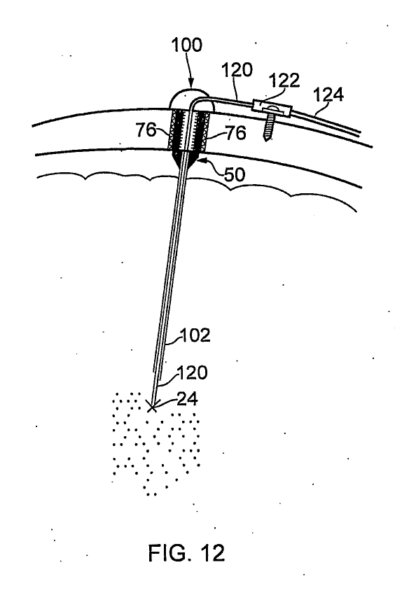

FIG. 12 illustrate a fine catheter inserted through the guide tube for delivery of therapeutic substances to a target region of the brain,

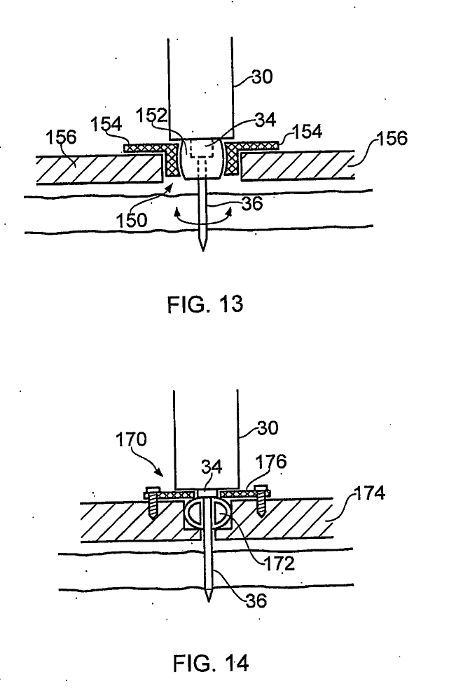

FIG. 13 illustrates an alternative, pivotable, skull mount,

FIG. 14 illustrates a further skull mount formed partially from skull bone,

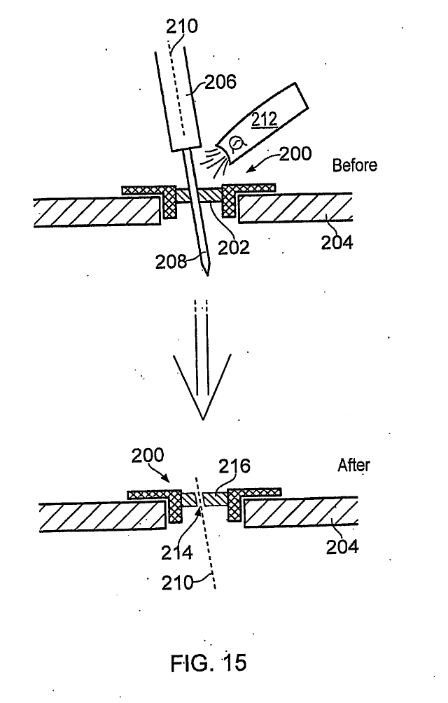

FIG. 15 illustrates a skull mount having an adhesive based alignment guide,

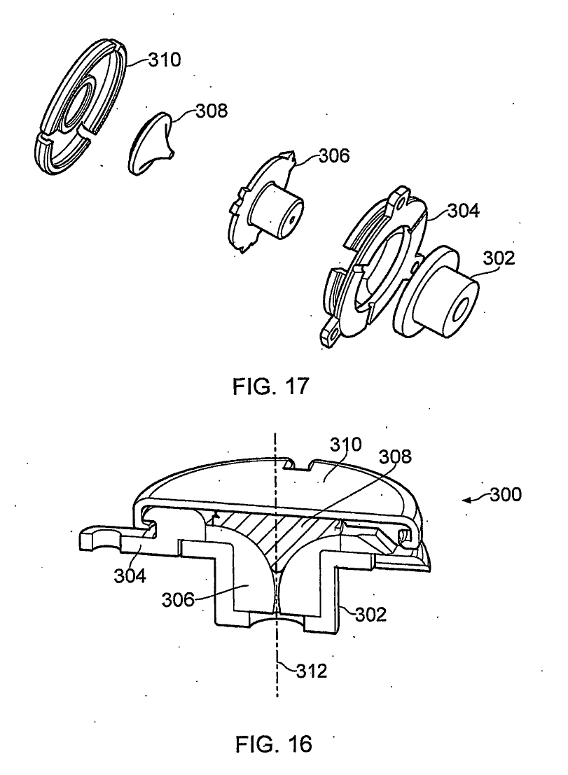

FIG. 16 illustrates a further skull mount of the present invention, and

FIG. 17 is an exploded view showing the components of the skull mount of FIG. 16.

In order to perform neurosurgery, the surgeon, in the first instance, identifies the position of the desired target or targets within the brain. Stereotactic localisation of a brain target or targets can be accomplished by securely fixing a stereotactic base ring to the subject's skull and identifying the position of the target using imaging techniques, such as magnetic resonance imaging (MRI). The position of the target can be identified in three dimensional co-ordinates by making measurements with reference to radio-opaque fiducials that are attached, in known positions, to the stereotactic base ring. The radio-opaque fiducials may be contained in what is termed a localiser box that is repeatably mountable to the stereotactic base ring.

After acquiring the necessary MRI data, the localiser box can be detached from the stereotactic base ring, which remains attached to the patient. A stereoguide can then be attached to the stereotactic base ring and used as a platform from which to guide neurosurgical instruments to the identified target(s). In is important to note that in such an arrangement the position of the radio-opaque fiducials of the localiser box and the position of the stereoguide are both known relative to the stereotactic base ring. This allows the stereoguide to guide instruments to the target co-ordinates identified from the MRI images. A stereotactic system of this type is commercially available from Elekta AB, Stockholm, Sweden.

Referring now to FIG. 1, a stereoguide 2 of the type described above is illustrated when attached to a stereotactic base ring 4 that is in turn securely attached (e.g. screwed) to the head 6 of a subject. The stereoguide 2 comprises an arced portion 8 that is attached to the stereotactic base ring 4 by rotatable mounts 10. A platform 12 is also provided that can be slid around the arced portion 8. The platform carries a first (upper) guide member 14 attached to the platform by a first slidable mount 16 and a second (lower) guide member 18 attached to the platform by a second slidable mount 20. The first and second guide members 14 and 18 are arranged such that they are aligned to provide an axis of insertion 22. Furthermore, the first and second slidable mounts 16 and 20 allow the radial position of the first and second guide members 14 and 18 to be adjusted without altering the defined axis of insertion. The platform 12 also be moved around the arced portion 8, and the arced portion 8 can be rotated relative to the base ring 4 using mounts 10, to alter the axis of insertion 22 as required.

It should be noted that the stereoguide also comprises scale markings (not shown) that provide an accurate measure of (a) the position of the first and second guide members 14 and 18 relative to the platform 12, (b) the angular position of the platform 12 relative to the arced portion 8 and (c) the rotational position of the arced portion 8 relative to the stereotactic base ring 4 (i.e. the angular orientation adopted by rotatable mounts 10). In this manner, it is possible to relate the orientation of the axis of insertion 22 and any positions measured relative to the guide members 14 and 18 to the stereotactic base ring 4 and hence to target(s), such as target 24, that have been identified by a surgeon from the acquired MRI images.

After a target has been identified, the surgeon selects a suitable axis of insertion that reaches that target and configures the stereoguide accordingly. It should be noted that selecting the axis of insertion is not typically an arbitrary choice but is chosen so as to minimise the impact of the procedure on the subject. For example, the axis of insertion may be selected so as to avoid major blood vessels in the brain and/or any critical brain regions as identified by the MRI imagery. The stereoguide 2 may thus be set to provide the required axis of insertion 22 to the target 24.

The first stage of the surgical procedure is to drill a hole in the skull of the subject 6. To drill such a hole, a cranial drill is inserted through the first and second guide members 14 and 18 of the stereoguide 2 and brought into contact with the skull along axis 22. A hole can then be drilled through the skull bone, the hole being aligned with the axis of insertion 22.

The next stage of the surgical procedure, which will be described in detail with reference to FIGS. 2 to 6, is to implant a skull mount within the hole using a skull mount insertion and alignment device.

Referring to FIG. 2, a skull mount insertion and alignment device 30 is illustrated. The device 30 comprises an elongate shaft 32 having a substantially circular cross-section. The distal end of the shaft 32 carries a protrusion 34 having a circular cross-section of smaller radius than the shaft 32. A screw thread is provided on the outer surface of the protrusion 34 for engaging the skull mount described below with reference to FIG. 3. A stiff wire 36 having a diameter of around 0.8 mm passes through the centre of the protrusion 34 and extends from the distal end of the protrusion by about 10-12 mm. The distal end of the wire 36 may, if required, be tapered to a point. The proximal end of the shaft 32 carries an end stop 38 having a marking 40 to identify the angular orientation of the alignment device 30. The centres of the shaft 32, protrusion 34, wire 36 and end stop 38 are all substantially aligned along a common central axis of rotation 42. A scale 33 is marked on the shaft 32 to provide a measure of the distance (y) between the end (reference) surface 35 of the shaft 32 and an associated mark formed on the stereoguide in which the device is mounted during use.

Referring to FIGS. 3a to 3c, a skull mount 50 is illustrated. In particular, FIG. 3a shows a side view of the skull mount and FIGS. 3b and 3c are cross-sectional views through the skull mount along the planes identified in FIG. 3a as I-I and II-II respectively. The skull mount 50 comprises an (upper) annular attachment portion 52 comprising a ring portion 54 defining a cavity 64 and having an outer threaded surface 56 and inner threaded surface 58. The skull mount 50 also comprises a (lower) cylindrical tapered portion 60 having a central aperture 62 formed therethrough. The cavity 64 and the inner threaded surface 58 are arranged to compliment the protrusion 34 of the alignment device 30 described above with reference to FIG. 2. Similarly, the aperture 62 is configured to allow the stiff wire 36 of the above described alignment device 30 to pass therethrough. In this manner, the skull mount 50 can be screwed on to the distal end of the alignment device 30.

Referring to FIG. 4, a skull mount 50 attached to the end of a skull mount insertion and alignment device 30 is illustrated when being inserted into a stereoguide 2. As illustrated, the distal end of the alignment device 30 which carries the skull mount can be passed though the first and second guide members 14 and 18 of the stereoguide. The skull mount 50 can thus be passed along the axis of insertion and located within the hole 60 that has been previously formed in the subject's skull.

FIG. 5 illustrates in more detail the skull mount 50 and the skull mount insertion and alignment device 30 after the skull mount 50 has been located within the hole formed in the subjects skull bone 70. In particular, it can be seen from FIG. 5 how the stiff wire 36 of the skull mount insertion and alignment device 30 passes along the axis of insertion 22 and performs the function of perforating the dura 72 and forming a passageway through the cortex 74 (which is typically 10-12 mm thick). The device 30 can thus be thought of as a cortical obturator dural perforator (CODP). Although perforating the dura may be performed using the skull mount insertion and alignment device 30, it is also possible to pierce the dura prior to such a procedure; this prior piercing of the dura (e.g. manually by a surgeon using a scalpel or the like) can help to ensure no blood vessels are ruptured during the surgical procedure. An adhesive 76 is also provided to securely fix the skull mount 50 to the skull 70. The adhesive 76 is allowed to cure whilst the skull mount insertion and alignment device 30 remains attached to the skull mount 50.

Referring now to FIG. 6, it is shown how the skull mount insertion and alignment device 30 can (after the adhesive 76 has cured) be unscrewed from the skull mount 50 and withdrawn back through the stereoguide 2. In this manner, it can be seen that the aperture provided through the skull mount 50 is then accurately aligned with the axis of insertion as defined by the stereoguide. The implanted skull mount 50 can thus be considered a tertiary guide member that can aid the guiding of instruments along the axis of insertion. It can also be seen in FIG. 6 that the upper surface of the skull mount 50 is substantially flush to the surface of the skull after implantation.

After implantation of the skull mount, a guide tube is implanted having a distal end that terminates just short of the required target area. A guide tube applicator and guide tube will now be described with reference to FIGS. 7 to 11

Referring to FIG. 7, a guide tube applicator 80 is illustrated. The guide tube applicator 80 comprises an elongate shaft 82 having a central hollow channel through which a guide wire 84 can be passed. The outer diameter of the shaft 82 is preferably the same as the outer diameter of the shaft 32 of the skull mount insertion and alignment device 30. A clamp 86 is provided at the proximal end of the applicator 80 to prevent unwanted axial movement of the guide wire 84 relative to the guide tube applicator 80. The distal end of the applicator 80 comprises a dome shaped recess 88 having a central linear bar 90. An aperture through the bar 90 is provided for the guide wire 84. The shape of the recess 88 and bar 90 are complimentary to the shape of the guide tube hub described in more detail with reference to FIG. 8.

Referring to FIG. 8, a guide tube 100 of known type is shown. The guide tube 100 comprises a length of tubing 102 having a hub 104 at its proximal end. The sides of the hub carry a screw thread 106 and the top surface 108 of the hub, which has a lip extending further radially than the screw thread 106, is dome shaped and has a slot 110 formed therein. The slot 110 also provides the opening via which the lumen of tubing 102 can be accessed. As mentioned above, the top surface 108 of the guide tube hub 104 can be received in the recess 88 of the guide tube applicator 80. The slot 110 of the hub is also arranged to engage the bar 90 of the guide tube applicator 80 thereby preventing relative rotation of the guide tube 100 and guide tube applicator 80 when mated.

FIG. 9 illustrates a guide tube 100 attached to the distal end of a guide tube applicator 80 prior to its insertion into the guide members of the stereoguide 2. The required length of the guide tube 100 and the length of the guide wire 84 that protrudes from the guide tube applicator 80 can be calculated relative to the top surface of the skull mount 50; this calculation can be performed using the reading taken from the scale 33 of the skull mount insertion and alignment device 30 during the process of inserting the mount 50 into the hole.

Referring to FIG. 10, the guide tube applicator 80 is fed through the first and second guide members of the stereoguide (only the second guide member 18 being shown in FIG. 10) towards the subject. The guide tube 100, which is stiffened by the guide wire 84, passes through the skull mount 50 and into the brain of the subject. The skull mount 50 also acts as a guide member and may thus be considered a third or tertiary guide member. The guide wire 84 and guide tube 100 are thus driven together through brain tissue along the axis of insertion with a high level of accuracy. In particular, the provision of the third guide member (which is also aligned with the axis of insertion as described above) provides accurate guiding in the immediate proximity of the brain thereby minimising the possibility of suboptimal guide tube placement.

It should also be noted that using the skull mount insertion and alignment device 30 that is described above also improves the accuracy of guide wire 84 and guide tube 100 insertion. This is because, as also mentioned above, device 30 forms a passageway through the cortex and may also pierce the dura. The dura is a tough membrane and the cortex is around 10-12 mm of relatively tough brain tissue. Inserting the guide wire 84 and guide tube 100 through the pre-formed passageway in the dura and cortex reduces any deflection away from the axis of insertion that could occur if the guide wire 84 alone was to be urged into the brain. Alternatively, the guide wire 84 can have a smaller diameter (thereby having a lower stiffness) than would be necessary if it was required to penetrate the dura and cortex.

Insertion continues until the hub 104 of the guide tube 100 makes contact with the skull mount 50. As described above with reference to FIG. 3, the skull mount includes a cavity 64 having a threaded wall 58. The hub 104 of the guide tube 100 is configured so that it can be screwed into cavity 64 of the skull mount. This is achieved by rotating the guide tube applicator 80. Once the hub 104 is screwed into place, the guide tube applicator 80 (including the guide wire 84) can be withdrawn back through the guide members of the stereoguide. As shown in FIG. 11, the skull mount 50 and guide tube 100 are then retained in the subject's skull.

Referring to FIG. 12, use of the above described implanted guide tube 100 for receiving a catheter 120 is illustrated. In particular, FIG. 12 shows a skull mount 50 secured in a skull hole by an adhesive 76. The guide tube 100 is screwed into the skull mount 50 and comprises a length of tubing 102 located along the axis of insertion and terminating just short of the required target 24. FIG. 12 also shows a catheter 120 that has been passed through the guide tube and is arranged to be of a length such that its distal end reaches the required target 24. The proximal end of the catheter 120 may be secured to the skull by a clip 122. The catheter 120 may also be in fluid communication with a drug delivery pump (not shown) via a wider bore supply tube 124. In this manner, the required therapeutic agent may be delivered to the target site 24 via catheter 120. To minimise the risk of infection passing the blood-brain barrier, the catheter 120 and guide tube 100 may be subcutaneously mounted and the supply tube 124 subcutaneously channeled to an implanted drug delivery pump. It should be noted at this point that the catheter 120 may be inserted through the guide tube without the use of a stereoguide and can thus be relatively easily replaced if necessary.

Referring now to FIGS. 13 and 14, alternative skull mounts suitable for use in the above described surgical procedure are illustrated.

FIG. 13 shows a skull mount insertion and alignment device 30 having a pivotable skull mount 150 attached to its distal end. The pivotable skull mount 150 comprises a truncated ball 152 having a cavity with an internal screw thread surface for receiving the protrusion 34 of the device 30 and a channel through which the stiff wire 36 of the device 30 passes. The pivotable skull mount 150 also comprises a casing or socket portion 154 for retaining the ball 152. The casing portion is suitable for insertion into a hole formed through the skull 156.

In use, the upper rim of casing portion 154 can be secured to the skull using adhesive or screws etc (not shown). The skull mount insertion and alignment device 30 may then be moved along the axis of insertion using the stereoguide and engaged with the truncated ball 152. As shown in FIG. 13, the channel through the truncated ball 152 becomes aligned with the axis of insertion as defined by the stiff wire 36 of the device 30. The ball 152 may then be locked in position relative to the casing portion 154; such locking may be permanent (e.g. adhesive) or releasable (e.g. by using releasable locking screws). This pivotable arrangement has several advantages. For example, it allows an axis of insertion to be used that deviates significantly from the skull normal. It can also simplify the skull mount insertion process and, if a releasable locking mechanism is used, allows subsequent angular adjustments to the axis of insertion.

FIG. 14 shows a skull mount 170 that is a variant to the skull mount 150 of FIG. 13 and is also suitable for use with the above described skull mount insertion and alignment device 30. The skull mount 170 comprises a truncated ball 172 retained within a cavity. The bottom and sides of the cavity are formed by a recess drilled in the skull bone 174. A plate 176 having a triangular cross-section aperture is placed over the recess and screwed to the skull thereby forming the top of the cavity. In this manner, a lower complexity skull mount may be provided, albeit with a requirement for the surgeon to provide a stepped recess in the skull 174. A threaded recess may also be provided on the internal surface of the channel formed through the ball 172 for mating with the skull mount insertion and alignment device.

Referring to FIG. 15, a further skull mount 200 is illustrated. The skull mount 200 comprises a layer of (uncured) UV curable adhesive 202 and is attached to a hole formed in the skull 204 (e.g. with adhesive or by a screw thread attachment). After skull mount attachment to the skull, an alignment instrument 206 comprising a protruding member 208 is passed along the required axis of insertion 210 and penetrates the layer of adhesive. An ultraviolet (UV) light source 212 is then used to cure the adhesive layer 202 with the alignment instrument in situ. The protruding member is formed from, or coated with, a material (e.g. a surfactant) that does not adhere to the cured adhesive. It is thus possible to retract the alignment instrument 206 after the adhesive layer 202 has been cured thereby providing an alignment guide in the form of an alignment channel 214 in a layer of cured adhesive 216; the alignment channel 214 being aligned with the axis of insertion 210.

Referring to FIGS. 16 and 17, a further skull mount 300 of the present invention is illustrated.

The skull mount 300 comprises a skull insert 302 and a retaining ring 304. The skull insert 302 is dimensioned so as to fit in a hole formed in the skull and has a protruding lip for engaging the outer surface of the skull around the periphery of the hole formed in the skull. The skull insert 302 is held in place by the ring 304 which can in turn be secured to the skull by bone screws. An elastomeric septum seal guiding member 306 fits within a cavity defined by the skull insert 302 and the retaining ring 304. The septum seal guiding member 306 includes an aperture that defines an axis of insertion 312. The septum seal guiding member 306 also provides a fluidic seal with a catheter or other neurosurgical instrument passed through its aperture along the axis of insertion 312. A cap 310 and a cap sealing bung 308 are also provided. The cap sealing bung 308 fits within, and forms a seal with, the septum seal guiding member 306 and is held in place by the cap 310 which is attachable to the retaining ring 304 by a snap fit. The skull mount 300 thus provides a sealed passageway into the brain for a catheter or electrode etc. Furthermore, appropriate alignment of the aperture of the septum seal guiding member 306 (e.g. using a skull mount alignment device) allows that member to provide a guiding function.

The above examples are directed to accurately inserting guide tubes through which catheters may then be passed for delivery of therapeutic substances (e.g. drugs) to the brain. The techniques and apparatus described above are, however, also applicable for inserting electrodes into the brain for deep brain stimulation. For example, the catheter 120 shown in FIG. 12 may be replaced with an electrode that is connected to a suitable power source. Alternatively, the guide wire 84 and guide tube 100 inserted into the brain by the guide tube applicator 80 as described with reference to FIGS. 7-10 may be left in place for DBS purposes. It is even possible for the guide tube to be omitted altogether and the guide tube applicator 80 as described with reference to FIG. 7 may be used to insert only a guide wire (e.g. guide wire 84) through the skull mount and into the brain. Furthermore, although the insertion of only one guide tube into a subject is described above, the technique may be repeated multiple time on a single subject to insert multiple guide tubes and/or electrodes to different target areas of the brain.

1. A skull mount attachable to a hole formed in the skull of a subject, the skull mount comprising an alignment guide defining an alignment axis along which neurosurgical instruments can be passed, characterised in that the skull mount, when attached to a hole in a skull, does not substantially protrude from the outermost surface of the skull and does not extend into the brain parenchyma.

2. A skull mount according to claim 1 that, when attached to a hole formed in the skull of a subject, is substantially flush to the outermost surface of the skull.

3. A skull mount according to any preceding claim suitable for long term, subcutaneous, implantation within a subject.

4. A skull mount according to any preceding claim comprising a member defining the alignment guide and a socket attachable to a hole formed in a subject's skull, wherein the member defining the alignment guide is retained by, and is moveable relative to, the socket.

5. A skull mount according to any one of claims 1 to 3 wherein the alignment guide comprises a member having a channel formed therethrough defining the alignment axis, wherein the orientation of the skull mount within a hole formed in the skull is set during attachment of the skull mount to the skull to align the alignment axis with the required axis of neurosurgical instrument insertion.

6. A skull mount according to any preceding claim wherein the alignment guide can be immobilised relative to the skull after implantation.

7. A skull mount according to any preceding claim that can be affixed to a hole formed in the skull of a subject with adhesive.

8. A skull mount according to any preceding claim comprising a recess that allows releasable attachment of the skull mount to a neurosurgical alignment instrument, wherein the surface defining the recess carries a screw thread for releasable attachment to a complimentary protrusion provided on an associated neurosurgical alignment instrument.

9. A neurosurgical alignment instrument for aligning a skull mount, the skull mount being attachable to a hole formed in the skull of a subject and including an alignment guide defining an alignment axis along which neurosurgical instruments can be passed, the instrument comprising;
an elongate shaft; and
an element protruding from the distal end of the elongate shaft for engaging and aligning the alignment guide of an associated skull mount;
characterised in that, when the instrument is engaged with a skull mount attached to a hole formed in the skull of a subject, the protruding element passes through the alignment guide of the skull mount and into the cortex of the subject's brain.

10. An instrument according to claim 9 wherein the protruding element comprises a wire that is substantially co-axial with longitudinal axis of the elongate shaft.

11. An instrument according to any one of claims 9 to 10 wherein, in use, the protruding element is arranged to penetrate 10 mm to 12 mm into the brain.

12. An instrument according to any one of claims 9 to 11 wherein an attachment member is also provided at the distal end of the elongate shaft, the attachment member being releasably engageable with an associated skull mount.

13. An instrument according to any one of claims 9 to 12 wherein a plurality of scale markings are provided on the elongate shaft.

14. Neurosurgical apparatus comprising;
a stereoguide for guiding neurosurgical instruments along a defined axis of insertion;
a skull mount according to any one of claims 1 to 8; and
a skull mount alignment instrument according to any one of claims 9 to 13 for aligning the alignment axis of the skull mount;
wherein, in use, the skull mount alignment instrument is carried by the stereoguide and aligns the alignment axis of the skull mount with the axis of insertion defined by the stereoguide.

15. An apparatus according to claim 14 comprising an applicator instrument for retaining a guide wire, wherein, in use, the applicator instrument is carried by the stereoguide and allows a guide wire to be passed through the alignment guide of an implanted skull mount and into the brain parenchyma, the stereoguide and the alignment guide of the skull mount acting to guide the guide wire along the defined axis of insertion.

16. An apparatus according to any one of claims 14 to 15 further comprising at least one of a guide wire, a catheter, a guide tube, an electrode and a biopsy needle.

17. A method for aligning a skull mount relative to a hole formed in a subject's skull, the skull mount comprising an alignment guide defining an alignment axis along which neurosurgical instruments can be passed, the method comprising the step of (i) using a stereoguide to align said alignment axis with a predetermined axis of insertion.

18. A method according to claim 17 wherein step (i) comprises the step of using a stereoguide that forms part of a stereotactic frame that is mounted to the subject's skull.

19. A method according to claim 17 wherein step (i) is preceded by a step of configuring the stereoguide to guide neurosurgical instruments along the predetermined axis of insertion.

20. A method according to claim 17 wherein step (i) is preceded by the step of determining the axis of insertion along which neurosurgical instruments are to be guided to a desired target in the brain parenchyma.

21. A method according to claim 17 in which step (i) comprises using the stereoguide to guide a neurosurgical alignment instrument along the predetermined axis of insertion, the neurosurgical alignment instrument comprising an elongate shaft and an element protruding from the distal end thereof, wherein step (i) comprises bringing the protruding element of the neurosurgical alignment instrument into engagement with the alignment guide of the skull mount, thereby aligning the alignment axis of the skull mount with the predetermined axis of insertion.

22. A method according to claim 21 in which the distal end of the protruding element of the neurosurgical alignment instrument is arranged to pass through the alignment guide of the skull mount, wherein step (i) comprises forcing the distal end of the protruding element through the subject's cortex.

23. A method according to claim 21 wherein step (i) comprises using the neurosurgical alignment instrument to carry a skull mount along the axis of insertion and into engagement with the hole formed in the subjects skull.

24. A method according to claim 17 comprising the step (ii) of fixing the orientation of the alignment axis of the alignment guide of the skull mount after step (i) has been performed.

25. A method according to claim 24 comprising the step (iii) of using the stereoguide to pass a guide wire through the alignment guide of the skull mount and along the predetermined axis of insertion into the brain parenchyma.

26. A method according to claim 25 wherein step (iii) comprises passing a guide wire inserted into a guide tube through the alignment guide of the skull mount and along the predetermined axis of insertion into the brain parenchyma.

27. A method according to claim 26 comprising the step (iv) of withdrawing the guide wire from the subject whilst leaving the guide tube in situ.

28. A method according to claim 27 comprising the step (v) of inserting at least one of an intraparenchymal catheter and an intraparenchymal electrode into the brain parenchyma through the guide tube.

29. A method according to claim 17 in which step (i) is preceded by the step of using a drill bit to drill a hole in the skull of the subject, wherein the stereoguide is used to pass the drill bit along the predetermined axis of insertion into contact with the subject's skull.
